(19) United States
(12) Patent Application Publication
(10) Pub. No.: US 2022/0396787 A1
(43) Pub. Date: Dec. 15, 2022

(54) METHOD FOR ENHANCED DIRECT DETECTION OF MICROBIAL ANTIGENS FROM BIOLOGICAL FLUIDS

(71) Applicant: ACUTIS DIAGNOSTICS, INC., East Northport, NY (US)

(72) Inventors: Mayilvahanan SHANMUGAM, Hicksville, NY (US); Mumtaz AKHTAR, Hicksville, NY (US); Marjorie BONHOMME, Hicksville, NY (US); Michael FICUILLI, Hicksville, NY (US); David GOLDBERG, Hicksville, NY (US); Wahid SARIJ, Hicksville, NY (US)

(73) Assignee: ACUTIS DIAGNOSTICS, INC., East Northport, NY (US)

(21) Appl. No.: 17/837,593

(22) Filed: Jun. 10, 2022

Related U.S. Application Data
(60) Provisional application No. 63/209,436, filed on Jun. 11, 2021.
(60) Provisional application No. 63/315,742, filed on Mar. 2, 2022.

(51) Int. Cl. C12N 15/10; C12Q 1/686; C12Q 1/6806; G01N 21/64; C07K 1/36

(52) U.S. Cl. CPC C12N 15/1048; C12Q 1/686; C12Q 1/6806; G01N 21/6428; C07K 1/36

(57) ABSTRACT

The present disclosure provides methods and compositions for determining the presence of microorganisms in biological fluids by processing extracellular vesicles. The fluids are treated with a composition comprising a pH buffer, a nonionic surfactant, a zwitterionic detergent, an anionic surfactant, and a reducing agent. This treatment solubilizes extracellular vesicles in the fluid, enhancing the ability of diagnostic tools to find the microorganisms. The extracellular vesicles may also be isolated before solubilizing.

### CROSS-REFERENCE TO RELATED APPLICATIONS

The present application claims the benefit under 35 U.S.C. § 119 of U.S. Provisional Patent Application Ser. No. 63/209,436, filed on Jun. 11, 2021, and U.S. Provisional Patent Application Ser. No. 63/315,742, filed on Mar. 2, 2022, each of which is incorporated by reference.

### BACKGROUND OF THE DISCLOSURE

### 1. Field of the Disclosure

The present disclosure provides methods and devices for taking patient samples and solubilizing microbial extracellular vesicles found therein in order to increase sensitivity of diagnostic methods for enhanced detection of infectious microorganisms. The methods may also include the isolation of the extracellular vesicles before the solubilization and detection.

### 2. Description of the Related Art

Invasion of a host's bloodstream by any microorganism is indicative of a serious medical condition. The presence of any microbe in the bloodstream may result in dysregulated host response, which in turn can result in a wide array of clinical abnormalities, including but not limited to, multiple organ failure, granuloma formation, neurologic impairment, and potentially even death. Early detection of microbial infection is critical for improved prognosis and patient survival.

Some microorganisms are more difficult to detect in the bloodstream or in other body fluids than others for many reasons, including intricate host immune evasion mechanisms and/or low bacteria titers in the blood (low bacteremia), to name a few. For example, Borrelia burgdorferi, the causative agent of Lyme disease, is difficult to detect in the bloodstream using currently available methodologies.

All three domains of life (Archaea, Bacteria and Eukarya) have cell types that are capable of producing extracellular vesicles (EVs) that can be involved in many different functions, including intercellular communication and transport of virulence factors. In bacteria, EVs contain both periplasmic and cytoplasmic components, and are derived from the cell's outer-most cytoplasmic membrane. Therefore, the exteriors of the vesicles have similar antigenic properties (e.g., lipids and outer membrane protein profiles) as the parental cells. Furthermore, EVs, sometimes referred to as outer membrane vesicles (OMVs) when produced by Gram-negative bacteria, are enriched with proteins, genetic material, and sometimes even toxins, and are released into the extracellular environment. Production and release of the vesicles are increased in response to stress or other chemical signals.

Because of the many different roles EVs can play in organism survival and host toxicity, they have high potential for use in the development of effective diagnostic tools. The pathogen-specific content encapsulated in the vesicles can serve as a means for the enhanced direct detection of multiple different microorganisms, including fastidious organisms such as B. burgdorferi that are typically able to avoid detection through traditional means. This method of detection is applicable to all pathogenic bacteria, fungi, parasites and viruses. Borrelia species have special glycolipids in their outer membrane that are particularly immunogenic. These glycolipids are part of lipid rafts involved in protein trafficking and signaling pathways. Cholesteryl 6-O-acyl-β-D-galactopyranoside (ACGal), cholesteryl-β-D-galactopyranosid (CGal), and mono-α-galactosyl-diacylglycerol (MGalD) are specific glycolipids found in Borrelia, Helicobacter, Mycoplasma, Anaplasma, and Brachyspira species.

EVs produced by these organisms contain these special glycolipids can be used as biomarkers for detection and diagnostic purposes. However, there are no currently available cost-effective and convenient devices for the capture or isolation of EVs, because EVs are heterogenous and their functions differ depending on the environment. Current EV isolation requires specialized equipment like ultra-centrifugation/density gradient or gel-filtration chromatography, which are time consuming, expensive and technically challenging methods.

### SUMMARY OF THE DISCLOSURE

The present disclosure provides a solubilization composition and methods for treating biological fluid samples so that infectious microorganisms can be detected more readily. The present disclosure provides a novel solubilizing composition that can be added to the biological fluid sample to solubilize the extracellular vesicles therein, thus enhancing the diagnostic methods for detecting the microorganisms. The methods include various centrifugation steps, chemical treatment, and other processes to achieve this result.

Accordingly, in one embodiment, the present disclosure provides a composition for solubilizing extracellular vesicles, comprising: a pH buffer; a nonionic surfactant; a zwitterionic detergent; an anionic surfactant; and a reducing agent.

The present disclosure also provides a method of determining the presence of a microorganism in a sample of a biological fluid, comprising the steps of: preparing or obtaining the sample of the biological fluid; centrifuging the sample to separate the fluid into a supernatant and a pellet; discarding the pellet; and solubilizing extracellular vesicles present in the supernatant by mixing the above-described composition with the supernatant to form a first mixture; and analyzing the first mixture to determine the presence of the microorganism.

The present disclosure also provides a method of determining the presence of a microorganism in a sample of a biological fluid, comprising the steps of: preparing or obtaining the sample of the biological fluid; centrifuging the sample to separate the fluid into a supernatant and a pellet; discarding the pellet; isolating extracellular vesicles present in the supernatant by mixing the supernatant with a sodium acetate solution to form a first mixture; centrifuging the first mixture to create a second pellet and a second supernatant; discarding the second supernatant; solubilizing extracellular vesicles present in the second pellet by mixing the above-described composition with the second pellet to form a second mixture; and analyzing the second mixture to determine the presence of the microorganism.

### BRIEF DESCRIPTION OF THE DRAWINGS

FIG. 1 is a schematic depiction of a first method of the present disclosure.

FIG. 2 is a schematic depiction of a second method of the present disclosure.

FIG. 3 is a schematic depiction of a third method of the present disclosure.

FIG. 4 is a schematic depiction of a fourth method of the present disclosure.

FIG. 5 is a schematic depiction of a fifth method of the present disclosure.

FIG. 6 is a schematic depiction of a sixth method of the present disclosure.

FIG. 7 is a schematic depiction of a seventh method of the present disclosure.

FIG. 8 is a schematic depiction of an eighth method of the present disclosure.

FIG. 9 is a schematic depiction of a ninth method of the present disclosure.

FIG. 10 is a schematic depiction of a tenth method of the present disclosure.

### DETAILED DESCRIPTION OF THE DISCLOSURE

Referring to the Figures, schematic drawings of the methods of the present disclosure are shown. The methods of the present disclosure provide for a rapid processing of a body fluid sample to determine the presence of a microbial antigen, and if so, what type of antigen it is. The methods of the present disclosure include treating the blood sample with a novel composition that includes a buffer such as Tris (tris(hydroxymethyl)aminomethane) and/or HEPES (4-(2-hydroxyethyl)-1-piperazineethanesulfonic acid), a nonionic surfactant such as Triton® X-100, a sulfobetaine zwitterionic detergent, e.g. CHAPS (3-[(3-cholamidopropyl)dimethylammonio]-1-propanesulfonate), an anionic surfactant such as sodium lauroyl sarcosinate, and a reducing agent such as dithiothreitol (DTT). The resulting composition is agitated and/or subjected to at least one centrifuge step to separate the components of the composition. In this way, the extracellular vesicles (EVs) of the sample are solubilized and optionally isolated. This enhances the ability of diagnostic methods to detect the present of infectious microorganisms.

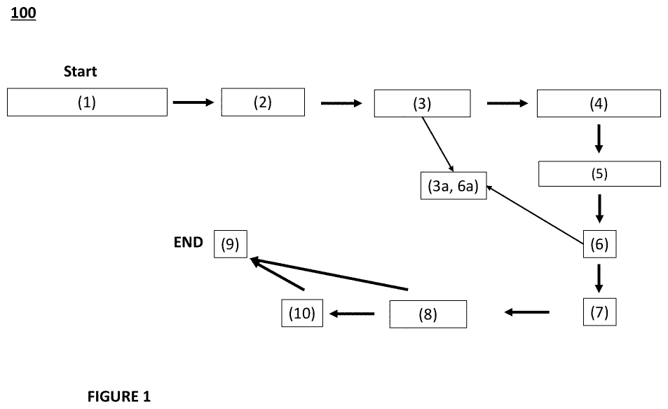

In FIG. 1, a first embodiment of the method of the present disclosure is shown, with reference numeral 100. A sample of bodily fluid is first taken from a patient (step 1) and can be reduced to 5-10 milliliters (step 2). The sample is then centrifuged for a first period of time (step 3). The first period of time can be approximately 10 minutes, or enough time to remove cellular components and comparatively large debris from the fluid sample. The pellet from the centrifuged sample is discarded (step 3a), and the supernatant filtered (step 4). The filtered supernatant is then solubilized (step 5) with the composition mentioned above and described in greater detail below. The treated sample is then spun or centrifuged a second time (step 6), for a second period of time. The second period of time can also be about ten minutes, or any period of time sufficient to separate any debris or particles from the sample. After the second spinning, the debris or pellet is discarded (step 6a), and the supernatant retained (step 7).

The supernatant is then treated with aptamers (step 8). The aptamers used here are selected to capture or enrich any microbial proteins or antigens in the sample. Aptamers are an affinity interaction-based macromolecule (single stranded DNA, RNA, or peptides) technology. They can have higher affinity for certain targets than antibodies, and are not as prone to issues with cross-reactivity like antibodies are. Target-specific aptamers can be synthesized after extensive screening from a library of aptamers. Attaching the target-specific aptamer molecules to beads (magnetic, latex, colloidal gold, etc.) allows for easy recovery of the aptamers bound to targets of interest.

After step 8, the sample can be run through a diagnostic tool (step 9) for detection and identification of the microbial agent. The diagnostic methods can include, but are not limited to, ELISA, Western Blot, high-resolution flow cytometry, mass spectrometry, and bead-based capture and detection. The treated supernatant from step 8 may optionally also be subjected to an enzymatic digestion (step 10) before being subjected to mass spectrometric analysis. Mass spectrometric analyses of proteins can involve the enzymatic digestion of target proteins into peptides. This allows for higher sensitivity and may allow for the detection of modified proteins that might be missed in a targeted whole protein method. A specialized enzyme class known as proteases are capable of breaking down proteins into peptides (for example, trypsin).

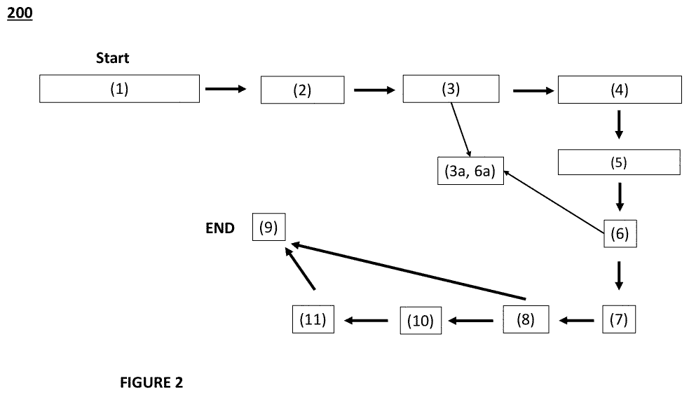

FIG. 2 is a diagram of a second method 200 of the present disclosure. Method 200 is almost identical to method 100, and its steps 1-10 are numbered accordingly. That is, step 1 of method 200 is identical to step 1 of method 100, step 2 of method 200 identical to step 2 of method 100, etc. The only difference between the two methods is that in method 200, there is an additional step after the enzymatic digestion (step 10). Method 200 includes an enrichment step 11, namely solid-phase extraction (SPE) and enrichment of the peptides in the treated liquid sample.

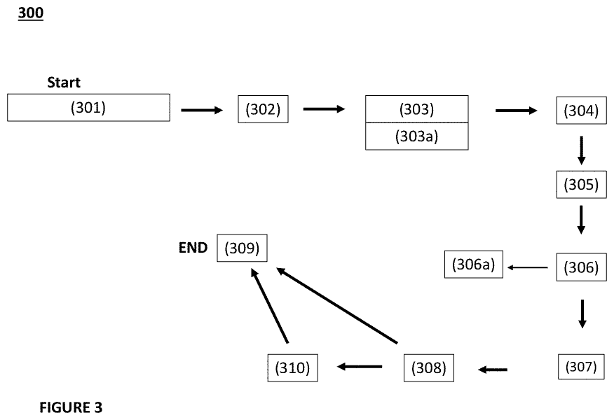

FIG. 3 is a diagram of a third method 300 of the present disclosure. In method 300, a sample of bodily fluid is first taken from a patient (step 301) and can be reduced to 5-10 milliliters (step 302). The sample is then centrifuged for a first period of time (step 303). The first period of time can be approximately 10 minutes, or enough time to remove cellular components and comparatively large debris from the fluid sample. Like in method 100, the pellet is discarded (step 303a). The supernatant is retained and subsequently filtered (step 304). The filtered supernatant is then treated with an alkali metal acetate solution such as sodium acetate (step 305).

Step 305 is advantageous because alkali metal acetate solutions such as sodium acetate neutralize the net negative charge of the phospholipids that make the vesicles, thus rendering them insoluble in water and causing them to crash or precipitate out of solution. This is faster than other vesicle purification methods, including differential centrifugation, ultracentrifugation, affinity chromatography and/or size-exclusion chromatography. Not only is it faster, it is also easily accomplished in a routine clinical laboratory, whereas the most common alternative, ultracentrifugation, is an expensive, time-consuming, and technically challenging procedure.

After step 305, the treated sample is then spun or centrifuged a second time (step 306), for a second period of time. The second period of time can also be about ten minutes, or any period of time sufficient to separate any debris or particles from the sample. After the second spinning, the supernatant is discarded (step 306a). The remaining pellet is solubilized with the solubilizing solution of the present disclosure (step 307). In the same way as in method 100, the solubilized sample is then treated with aptamers for specific capture of the target (step 308). After this step, the sample can be run through a diagnostic tool (step 309) for detection and identification of the microbial agent. The treated sample from step 308 may optionally also be subjected to an enzymatic digestion (step 310) before diagnostics.

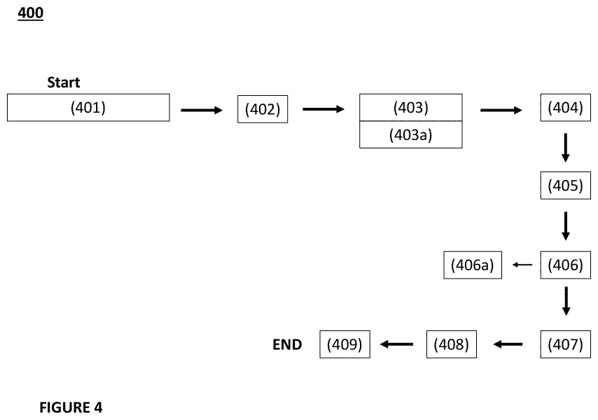

FIG. 4 is a diagram of method 400. Steps 401 through 406a are the same as their similarly numbered steps in method 300 (step 401 is identical to step 301, step 402 to step 302, etc.). In method 400, after the second centrifuge step 406, the sample pellet is subjected to solubilization followed by liquid-liquid extraction (step 407). After this, the liquid concentrate from step 407 is removed, dried down, and then reconstituted in a solution appropriate for targeted analysis (step 408), and subjected to the diagnostic methods previously discussed (step 409). Liquid-liquid extraction is a reliable method for extraction of glycolipids, which can be targeted in method 400.

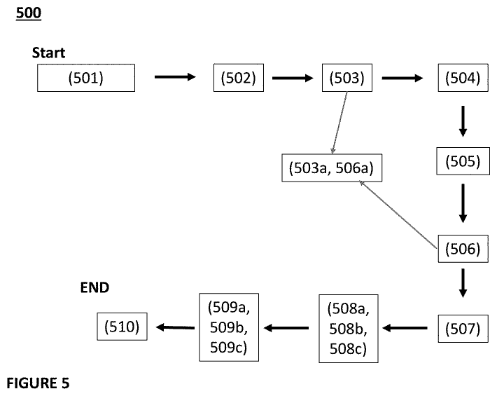

Method 500 is shown in FIG. 5. Steps 501-507 of this method, including steps 503a and 506a, are identical to steps 1 through 7 in method 100, including steps 3a and 6a. However, instead of having the target analyte(s) extracted from the supernatant by specific aptamers for direct detection of the target analyte (steps 8-10 in method 100), all protein contents of the supernatant of the solubilized EVs produced in method 500 are immobilized to a plate surface and washed (step 508a) for indirect detection of the target analyte(s). Target-specific, modified aptamers are then incubated with the bound samples and allowed to form analyte-aptamer complexes (step 508b). Unbound aptamers are washed away and discarded (step 508c). Specimens are then prepared for analysis by adding fluorescently labeled probes and allowing them to hybridize with the remaining bound aptamers (step 509a). Excess probes are then washed away (step 509b), and the hybridized probes get released from the aptamers into the supernatant through photolysis (step 509c). The collected fluorescent supernatant can then be subjected to high-resolution flow cytometry (step 510) for indirect detection of the target analyte(s).

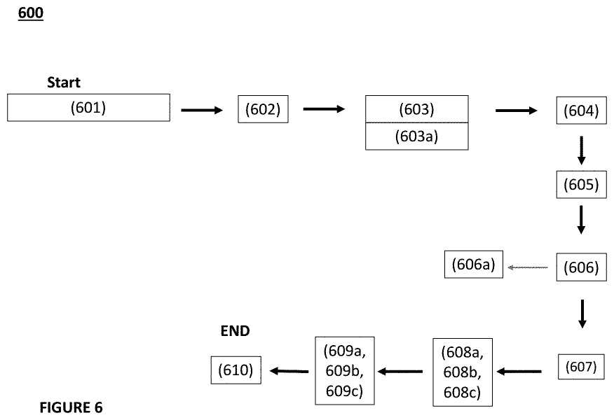

FIG. 6 shows method 600, which involves a step involving the isolation of EVs from samples prior to solubilization. Steps 601-606, including 603a and 606a, of this method are identical to steps 301-306 as outlined above for method 300, including steps 303a and 306a. Solubilized vesicles are then subjected to indirect detection of the target analyte(s) as described in steps 508 to 510 in method 500, and accordingly are numbered with reference numerals 608 through 610.

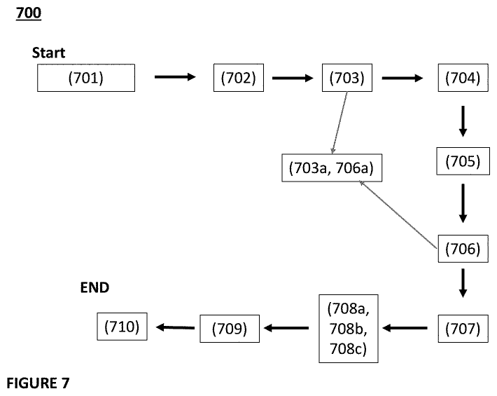

The procedure for method 700 (FIG. 7) mirrors the procedure outlined in method 500 for the first 8 steps of the procedure, with corresponding numerals 701 through 708. However, in method 700, the bound aptamer sequences are extracted from the aptamer-analyte complexes (step 709) and then subjected to amplification and detection using a polymerase chain reaction (PCR) derived technology for indirect detection of the target protein analyte(s) (step 710).

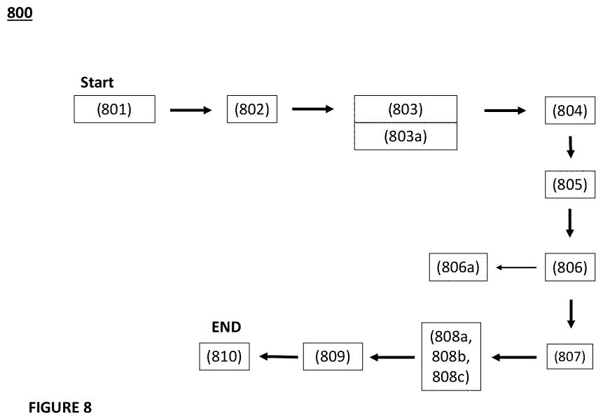

Method 800 (outlined in FIG. 8) is another procedure for the indirect detection of a target analyte(s) using PCR amplification of specific aptamers. The difference between methods 700 and 800 is that method 800 involves the isolation of EVs prior to solubilization. The first eight steps (801-808) of this method mirror steps 601 through 608 of method 600, and the last two (809-810; aptamer extraction and amplification) mirror the last two steps described in method 700, i.e. steps 709 and 710.

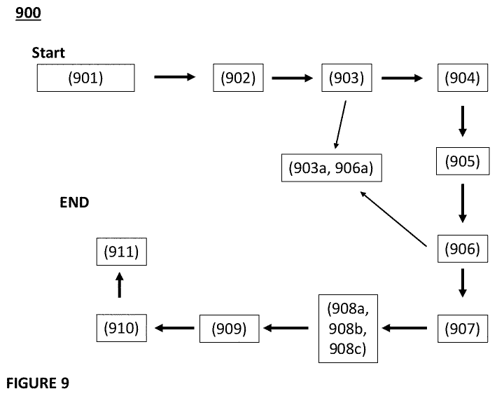

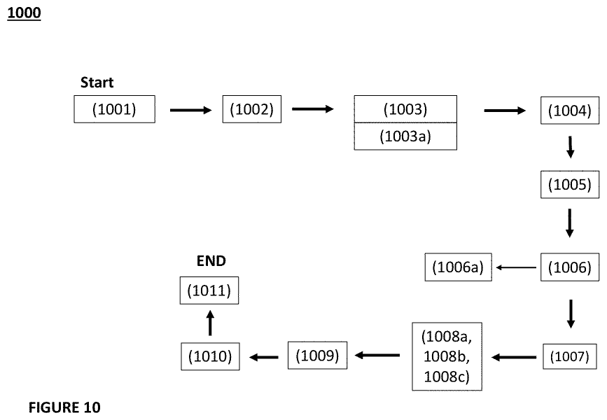

Methods 900 and 1000 (FIGS. 9 and 10, respectively) are also indirect methods for detection that involve nucleic acid amplification of analyte-specific aptamers that have been bound to captured target molecules. However, the difference between these two methods and the procedures described in methods 700 and 800 is that the amplification of the aptamer sequences is not the end point of the method. In methods 900 and 1000, the amplicons generated during PCR are detected using mass spectrometric technologies, such as MALDI-TOF. Method 900 is identical to method 700 with the addition of the final mass spectrometry step (step 911), and method 1000 is identical to method 800 with the addition of a final mass spectrometry step (step 1011).

Table 1 below shows the types of ingredients (Table 1) and amounts for the solubilization composition. Table 2 lists specific suitable candidates for the ingredients of claim 1.

TABLE 1

| Component | Concentration | |
|---|---|---|
| Buffer (pH 7-8) | 10-50 | mM |
| Non-ionic detergent | 1-4% | (v/v) |
| Zwitterionic surfactant | 10-200 | mM |
| Anionic surfactant | 1-10% | (w/v) |
| Reducing reagent | 0.1-1 | mM |

TABLE 2

| Component | Concentration | |
|---|---|---|
| Tris or HEPES | 10-50 | mM |
| Triton ® X-100 | 1-4% | (v/v) |
| CHAPS | 10-200 | mM |
| Sarkosyl | 1-10% | (w/v) |
| DTT | 0.1-1 | mM |

While the present disclosure has been described with reference to one or more exemplary embodiments, it will be understood by those skilled in the art that various changes may be made and equivalents may be substituted for elements thereof without departing from the scope of the present disclosure. For any ranges described above, the present specification contemplates that range, as well as any subranges therebetween. For example, if the present specification recites a range of 30 to 60 seconds, the present disclosure also contemplates 35-55 seconds, 40-50 seconds, 30-55 seconds, etc. In addition, many modifications may be made to adapt a particular situation or material to the teachings of the disclosure without departing from the scope thereof. Therefore, it is intended that the present disclosure not be limited to the particular embodiment(s) disclosed as the best mode contemplated, but that the disclosure will include all embodiments falling within the scope of the appended claims.

What is claimed is:

1. A reagent composition for solubilizing extracellular vesicles, comprising:
a pH buffer;
a nonionic surfactant;
a zwitterionic detergent;
an anionic surfactant; and
a reducing agent.

2. The composition of claim 1, wherein the pH buffer is selected from the group consisting of Tris, HEPES, and a combination thereof.

3. The composition of claim 1, wherein the nonionic surfactant has a hydrophilic polyethylene oxide chain and an aromatic hydrocarbon lipophilic or hydrophobic group.

4. The composition of claim 1, wherein the zwitterionic detergent is CHAPS.

5. The composition of claim 1, wherein the anionic surfactant is sodium lauroyl sarcosinate.

6. The composition of claim 1, wherein the reducing agent such is dithiothreitol (DTT).

7. The composition of claim 1, wherein the composition comprises:
between 10 to 50 millimolar by concentration of the pH buffer;
between 1% to 4% by volume of the nonionic surfactant;
between 10 to 200 millimolar by concentration of the zwitterionic detergent;
between 1% to 10% weight by volume of the anionic surfactant; and
between 0.1 to 1 millimolar by concentration of the reducing agent.

8. A method of determining the presence of a microorganism in a sample of a biological fluid, comprising the steps of:
preparing or obtaining the sample of the biological fluid;
centrifuging the sample to separate the fluid into a supernatant and a pellet;
discarding the pellet;
solubilizing extracellular vesicles present in the supernatant by mixing the composition of claim 1 with the supernatant to form a first mixture; and
analyzing the first mixture to determine the presence of the microorganism.

9. The method of claim 8, further comprising the step of, after the solubilizing step and before the analyzing step, centrifuging the first mixture to form a second pellet and a second supernatant.

10. The method of claim 9, further comprising the step of, after the second centrifuging step and before the analyzing step, treating the second mixture with a composition comprising aptamers.

11. The method of claim 10, further comprising the step of, after the treating step, enriching peptides in the second mixture.

12. The method of claim 8, further comprising the steps of, after the solubilizing step and before the analyzing step:
intubating modified aptamers with the first mixture to form a second mixture;
adding fluorescently labeled probes to the second mixture; and
hybridizing the fluorescent probes with aptamers.

13. The method of claim 8, further comprising the steps of, after the solubilizing step and before the analyzing step:
intubating modified aptamers with the first mixture to form a second mixture;
extracting bound aptamer from the second mixture; and
subjecting the second mixture to a polymerase chain reaction (PCR) derived technology for indirect detection of a target protein analyte(s).

14. The method of claim 13, further comprising the steps of, after an amplification of the second mixture during the subjecting step:
generating a third mixture of amplicons generated during PCR; and
subjecting the third mixture to mass spectrometric detection.

15. A method of determining the presence of a microorganism in a sample of a biological fluid, comprising the steps of:
preparing or obtaining the sample of the biological fluid;
centrifuging the sample to separate the fluid into a supernatant and a pellet;
discarding the pellet;
isolating extracellular vesicles present in the supernatant by mixing the supernatant with a sodium acetate solution to form a first mixture;
centrifuging the first mixture to create a second pellet and a second supernatant;
discarding the second supernatant;
solubilizing extracellular vesicles present in the second pellet by mixing the composition of claim 1 with the second pellet to form a second mixture; and
analyzing the second mixture to determine the presence of the microorganism.

16. The method of claim 15, further comprising the step of, after the solubilizing step before the analyzing step, treating the second mixture with a composition comprising aptamers.

17. The method of claim 15, further comprising the steps of, after the solubilizing step and before the analyzing step:
performing liquid-liquid extraction on the second mixture to extract glycolipids present in the second mixture, to form a dry concentrate; and
reconstituting the dry concentrate.

18. The method of claim 15, further comprising the steps of, after the solubilizing step and before the analyzing step:
intubating modified aptamers with the first mixture to form a second mixture; and
adding fluorescently labeled probes to the second mixture; and
hybridizing the fluorescent probes with aptamers.

19. The method of claim 14, further comprising the steps of, after the solubilizing step and before the analyzing step:
intubating modified aptamers with the first mixture to form a second mixture;
extracting bound aptamer from the second mixture; and
subjecting the second mixture to a polymerase chain reaction (PCR) derived technology for indirect detection of a target protein analyte(s).

20. The method of claim 19, further comprising the steps of, after the amplification of the second mixture during the subjecting step:
generating a third mixture of the amplicons generated during PCR; and
subjecting the third mixture to mass spectrometric detection.
